(19) United States
(12) Patent Application Publication
(10) Pub. No.: US 2019/0039269 A1
(43) Pub. Date: Feb. 7, 2019

(54) METHOD FOR PREPARING DOUBLE-NETWORK HYDROGEL TUBE WITH COMPLEX STRUCTURE

(71) Applicant: Lanzhou Institute of Chemical Physics, Chinese Academy of Sciences, Lanzhou (CN)

(72) Inventors: Feng ZHOU, Lanzhou (CN); Shuanhong MA, Lanzhou (CN); Weimin LIU, Lanzhou (CN)

(73) Assignee: Lanzhou Institute of Chemical Physics, Chinese Academy of Sciences, Lanzhou (CN)

(21) Appl. No.: 15/868,151

(22) Filed: Jan. 11, 2018

(30) Foreign Application Priority Data
Nov. 15, 2016 (CN) 201611001321.9

(51) Int. Cl. B29C 41/00; C08F 220/56; C08F 220/28; C08J 3/24; B29C 41/14

(52) U.S. Cl. CPC B29C 41/003; C08F 220/56; A61L 29/145; C08J 3/246; B29C 41/14; C08F 220/28

(57) ABSTRACT

The present invention discloses a method for preparing a double-network hydrogel tube with a complex structure. Iron wires of different sizes are mechanically polished, arranged in different manners, and then immersed into a monomer prepolymer solution, or a monomer prepolymer is poured into a container containing iron wires of different arranged shapes to conduct a polymerization reaction, such that a uniform primary-crosslinked single-network hydrogel film can be rapidly grown on the surface of each iron wire. The hydrogel film is then immersed in a secondary-crosslinking solution for secondary crosslinking to build a double-network hydrogel film. After immersion processing, the wires are drawn out to obtain high-strength hollow double-network hydrogel tubes of different shapes. The hydrogel tube has a diameter of 10 micrometers to a few millimeters, the shape of the tube inner structure is highly controllable, and the tensile strength of the hydrogel tube can be up to 2 MPa.

This application claims priority to Chinese application number 201611001321.9, filed 15 Nov. 2016, with a title of METHOD FOR PREPARING DOUBLE-NETWORK HYDROGEL TUBE WITH COMPLEX STRUCTURE. The above-mentioned patent application is incorporated herein by reference in its entirety.

### TECHNICAL FIELD

The present invention relates to a method for preparing a double-network hydrogel tube with a complex structure.

### BACKGROUND

A hydrogel tube can act as a novel implantable biological catheter, and plays a very important role in biological clinical medicine. Firstly, the hydrogel tube can be used as an in vitro implanted transferring catheter for human. For example, it can be used for transfer of fluid-like food and drug; in vitro excrement; artificial insemination and the like. During the application, the excellent low-friction property of hydrogel provides a protection for transfer of the fluid-like food and in vitro excrement; and the excellent stimulate responsiveness of the hydrogel provides the possibility of controllable transfer of drug. Therefore, it is of extremely important scientific and technological significance to develop a variety of general-purposed biological 3D hydrogel tubes for fluid transferring.

Furthermore, the hydrogel tube can be used as an artificial blood vessel to achieve blood transferring. In the research of biological catheters, it is more challenging to develop a safe and reliable artificial blood vessel, which is directly related to the national health level. According to statistics, every year more than 600,000 people worldwide need revascularization operation, and all countries of the world have made significant investment in research and development in this regard. Based on this, there are many reports for synthesizing blood vessels worldwide, and representative materials for preparing blood vessels includes: nylon, polyester, Teflon, natural mulberry silk and the like, and the preparation methods are divided into knitting, weaving and machine knitting. The artificial blood vessel which has been put into clinical application currently is Core-Tex made from polymer polytetrafluoroethylene. Although such polymer materials have better strength, the tensile properties thereof are poor. In the actual clinical trial, when the blood vessel is resected, it is very difficult to stretch and re-suture the remaining blood vessel. Although the vascular stretchable length varies depending on different diseased sites, the maximum stretched length is no more than 1 cm. However, surgeries for arteriosclerosis, aneurysm and the like almost all need to resect more than 1 cm of the blood vessel at the affected part. Therefore, during the treatment of similar diseases, it is typically necessary to use an artificial blood vessel to replace the resected part, or to reconstruct a branch vessel. The vascular transplantation means currently used in the medical field is vascular bypass, but the commonly-used plastic blood vessel is relatively harder and has poor flexibility, and the surface layer thereof is easily susceptible to formation of hemagglutination, and thus the bypass surgery is particularly prone to failure. Artificial blood vessels of less than 3 mm have not yet been commercialized on a large scale internationally, and thus it becomes a worldwide problem in the field of biomedical materials how to produce small-caliber blood vessels. Moreover, the current clinically-used blood vessel, either made of polymer substrate or plastic substrate, has a surface with poor water wettability, and thus it needs to modify the tube wall to improve its surface wettability for use.

The preparation method of a tubular body has undergone many times of technological changes. Initially, people sleeved two hollow tube-shaped molds with different diameters on each other, such that a tubular void was left between the outer wall of the mold with the smaller tube diameter and the inner wall of the mold with the larger tube diameter, and a prepolymer solution was casted into the void to obtain a hollow tubular body after a series of operations such as modeling, mold removing and the like. Such a traditional tube preparing process has great limitations. For example: it must be ensured that the sleeved tubes are highly concentric, otherwise a phenomenon of “core shift” will occur and causes a prepared tubular body with uneven thickness; in the preparation process it often needs to use operations such as heating, capping and the like, and thus the process is complex and the preparation process is energy-consuming and time-consuming; furthermore, a tubular body of branched or complex structure cannot be prepared through this method; and since such a method is too mechanical, it already has little use during industrial production. Currently, the production of tubular body mostly relies on the screw extrusion molding technology, the slurry for which the technology is used being a thermoplastic resin, a thermosetting resin and a rubber thereof and having been produced industrially in a large scale. Based on this preparation technology, the resulting polymer catheter (a rubber tube, a resin tube, a plastic tube) can be directly used as a fluid transferring channel and has been widely used in industry and daily life, but it is still of great challenges to use the polymer catheter as an artificial medical catheter in the biomedical field. A biomedical catheter must have physical and chemical properties similar to the environment of human extracellular matrix, and the specific requirements are as follows: 1. the materials used for preparation must have great biocompatibility; 2. the prepared biological catheter has great hydrophilicity and an excellent lubrication effect; 3. for safety, the prepared biological catheter should have good strength; 4. the prepared biological catheter has a certain ion permeability, such that it cannot only achieve fluid transferring, but also conduct exchanges of mass and energy with the external environment [U.S. Pat. No. 4,392,848, patent title: Catheterization]; 5. during the long-term use in the physiological environment, the catheter should have a certain chemical stability. Thus it can be seen that, it is very difficult to directly use a polymer catheter produced by a conventional technology as the artificial medical catheter and a surface modification treatment is required [U.S. Pat. No. 1,265,505. Coated catheters. National patent development corp. 31 Jul. 1969 [13 Aug. 1968], No. 38431/69]]. There are already patents which point out that a surface-grafted hydrogel [Patent No.: CA2211643, patent title: Hydrogel coatings containing commingled structurally dissimilar polymer hydrogels] can effectively improve the hydrophilicity, lubrication effect and biocompatibility of the surface of a disposable catheter, and it is hopeful to use the modified catheter as the artificial medical catheter; moreover, there are patent reports [Patent No.: US20150258247, EP2879729, CA2880526, patent title: self-lubricated catheters] which points out that the surface-modifying hydrophilic polymer coating can also effectively improve the physical and chemical properties on the surface of the catheter, and it is hopeful to use the modified catheter as the artificial blood vessel.

The surface-modified catheter has displayed outstanding application potentials in the medical field, but still has some limitations, and the specific analysis is as follows: 1. during the use process, friction and shear frequency occur between the modified catheter and surrounding tissues, which requires an ultra-strong binding force between the coating and the substrate catheter. Regardless of whether the catheter is used as a short-term fluid transferring channel or as a long-term tissue substitute (artificial blood vessel), a serious medical accident will be caused once the surface coating of the modified catheter is peeled off. 2. Surface modification can only improve the properties of the catheter surface, the inner wall of the integrally-formed catheter still has no ion permeability, and it is difficult to conduct exchanges of mass and energy between the catheter implanted in the human body environment and the extracellular matrix environment.

There are already published literatures in which it is also possible to obtain a hollow polymer catheter by a technology of extrusion molding through a spinning nozzle consisting of two concentric tubes, and it is hopeful to use the catheter as the artificial blood vessel. However, in this patent, it is necessary to use a specific polymer solution, and the polymer catheter is only obtained after the specific polymer solution undergoes a series of complex process treatments such as dry spinning and solidifying, and thus it does not have general applicability.

Based on this, it is quite challenging to develop a homogeneous catheter having an environment similar to that of the human extracellular matrix. The polymer hydrogel has physical and chemical properties similar to those of natural extracellular matrix, and is increasingly becoming the research focus of materials scientists. The use of an all-hydrogel catheter as a microfluidic channel facilitates full incubation of the implanted cells. The inner portion of the hydrogel tube can achieve a smooth fluid transferring, and the network structure on the tube wall of the hydrogel tube provides a guarantee for nutrient transfer, gas exchange, and removal of hazardous substances. Therefore it is hopeful to use these all-hydrogel catheters directly as artificial catheters. The research and development process of the artificial biomedical catheter will be greatly promoted if an all-hydrogel catheter can be developed. However, it is found through investigate and survey that currently there is still no patent on preparation technology for hollow hydrogel tube. Recently, the appearance of 3D bio-printing technology has made it possible to prepare artificial hydrogel tubes. However, the printing technology has relatively high requirements on the rheological properties of raw materials and the types of printing slurries that can be selected are relatively limited [the literature (Biomaterials, 2015, 61, 203-215) pointed out that a hollow hydrogel tube can be prepared by the bio-3D printing technology, but this system is only suitable for sodium alginate and calcium ions and thus has no general applicability], the printing accuracy is often poor and the prepared hydrogel tube has a relatively lower strength; the solidifying processes mostly use UV or heating means which have great harm to the cell; and thus an array of hydrogel tubes with complex structures cannot be printed. Therefore, it is still in the infancy stage to prepare a hollow hydrogel tube through 3D bio-printing and use it as the artificial medical catheter.

In view of the above, it is a scientific problem in this field whether a novel in vitro implanted transferring catheter having physical and chemical properties similar to those of natural extracellular matrix; whether an artificial medical catheter can be prepared by replacing the modified catheter with an all-biopolymer material to improve the compatibility of the catheter; and how to make a high-strength stretchable catheter of a millimeter-grade size or a lower size.

### SUMMARY

An objective of the present invention is to provide a method for preparing a double-network hydrogel tube with a complex structure and high strength.

As a three-dimensional network structural material with high water content, high elastic deformation, high strength and good biocompatibility, the hydrogel has physical and chemical properties similar to those of natural extracellular matrix and thus can be used as the material of choice for the research and development of artificial medical catheter.

In the present invention, iron wires of different sizes are mechanically polished, arranged in different manners, and then immersed into a monomer prepolymer solution, or a monomer prepolymer is poured into a container containing iron wires of different arranged shapes to conduct a polymerization reaction, such that a uniform primary-crosslinked single-network hydrogel film can be rapidly grown on the surface of each iron wire; the hydrogel film is immersed in a secondary-crosslinking solution for secondary crosslinking to obtain a double-network hydrogel film; and after immersion processing, the wires are drawn out to obtain high-strength hollow double-network hydrogel tubes of different shapes.

A method for preparing a double-network hydrogel tube with a complex structure is provided, where this method includes the following sequential steps:

1) immersing polished iron wires of different sizes into a monomer prepolymer solution, or pouring a monomer prepolymer solution into a container containing iron wires of different arranged shapes to conduct a polymerization reaction, such that a uniform chemically-crosslinked hydrogel layer is formed on the surface of each iron wire, aging, and then washing by soaking in pure water to obtain a primary-crosslinked single-network hydrogel film; and

2) immersing iron wires having grown hydrogel films in a secondary-crosslinking solution selected from an aqueous solution of calcium ions, an aqueous solution of magnesium ions, an aqueous solution of ferric ions, or an aqueous solution of tannic acid for 5 min to 20 h, and then drawing the iron wires out to obtain high-strength double-network hydrogel tubes of different shapes.

The monomer prepolymer solution as described in the present invention consists of a monomer, an initiator, an crosslinking agent, an aqueous polymer or biomacromolecule, and high-purity deionized water; wherein the monomer is one or two of (meth)acrylic acid, acrylamide, hydroxyethyl (meth)acrylate, polyoxyethylene methacrylate, N-isopropylacrylamide, methacrylic sulfonate, chitosan (meth)acrylate, chitosan (meth)acrylate, dimethylaminoethyl methacrylate, sodium alginate methacrylate, methylacryloyl ethylcarboxybetaine, and methylacryloyl ethylsulphobetaine; the initiator is potassium persulfate or ammonium persulfate; the crosslinking agent is N,N′-methylene bisacrylamide or (poly)ethylene glycol di(meth)acrylate; the aqueous polymer is polyvinyl alcohol, polyethylene glycol or polyvinylpyrrolidone; and the biomacromolecule is bovine serum albumin, collagen or polypeptide.

In the monomer prepolymer solution of the invention, the mass fraction of the monomer, the initiator and the crosslinking agent is from 5% to 15%, the molar ratio of the three is 500-1000:1:0.5, the mass fraction of the aqueous polymer is 5-10%, and the balance is deionized water.

In the monomer prepolymer solution of the invention, the mass fraction of the monomer, the initiator and the crosslinking agent is from 5% to 15%, the molar ratio of the three is 500-1000:1:0.5, the mass fraction of the biomacromolecule is 0.1-1%, and the balance is deionized water.

In the present invention, the iron wires are arranged in various ways and may be a single one, arranged in multiple rows, cross-arranged or arranged in an array, so as to obtain a hydrogel tube with a complex shape, and the diameter of the iron wire is 10 μm to 3 mm.

The polymerization time of the present invention is 1 to 30 min and the polymerization temperature is 10° C. to 30° C.

The secondary crosslinking solution of the present invention has a concentration of 0.1-0.6 mol/L.

The hydrogel tube prepared by the present invention has a tube diameter of 10 micrometers to a few millimeters, the shape of the tube inner structure is highly controllable, the tensile strength of the hydrogel tube can be up to 2 MPa, which is 2-5 times larger than the original one.

The method of the present invention has a low cost and a simple process, and is suitable for industrialized production, and thus has potential application values in the field of microfluids and biomedicine.

### BRIEF DESCRIPTION OF THE DRAWING

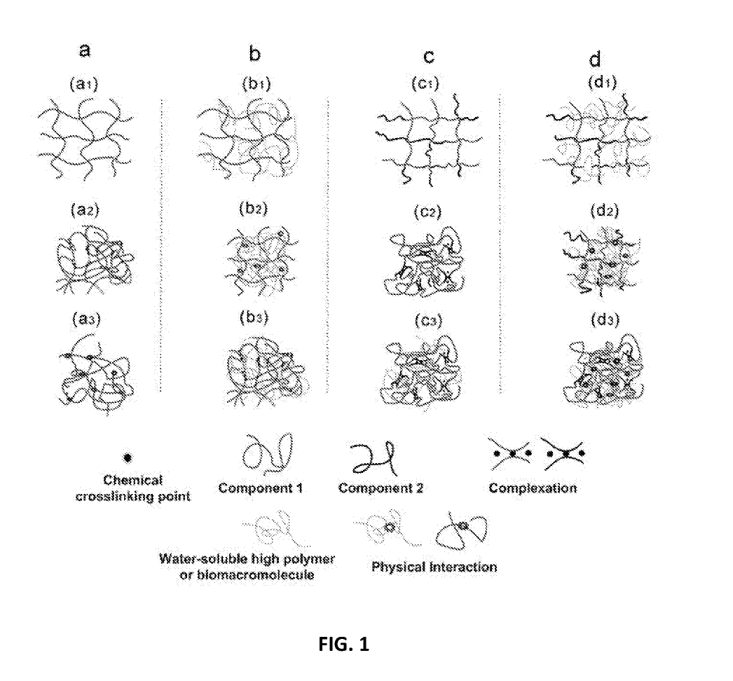

FIG. 1 is a schematic table of preparations of materials of a double-network hydrogel tube; including the following:
item a1 is a one-component chemically-crosslinked network hydrogel tube;
item a2 is a multi-network hydrogel tube in which the one-component (two-component and multi-component) chemical crosslinking and physical crosslinking cooperate with each other;
item a3 is a multi-network hydrogel tube in which the one-component (two-component and multi-component) chemical crosslinking cooperates with physical interaction;
item b1 is a composite network hydrogel tube in which one component (two components and multiple components) chemical crosslinking and polymer chains physically pass throughout;
item b2 is a multi-network hydrogel tube in which the one-component (two-component and multi-component) chemical crosslinking cooperates with physical interaction;
item b3 is a multi-network hydrogel tube in which the one-component (two-component and multi-component) chemical crosslinking/physical crosslinking/polymer chains physically pass throughout;
item c1 is a two-component and multi-component chemically-crosslinked network hydrogel tube;
item c2 is a multi-network hydrogel tube in which the one-component (two-component and multi-component) chemical crosslinking and physical crosslinking cooperate with each other;
item c3 is a multi-network hydrogel tube in which the one-component (two-component and multi-component) chemical crosslinking/physical crosslinking/polymer chains physically pass throughout;
item d1 is a composite network hydrogel tube in which one component (two components and multiple components) chemical crosslinking and polymer chains physically pass throughout;
item d2 is a multi-network hydrogel tube in which the one-component (two-component and multi-component) chemical crosslinking cooperates with physical interaction; and
item d3 is a multi-network hydrogel tube in which the one-component (two-component and multi-component) chemical crosslinking/physical crosslinking/physical interaction cooperate with each other.

### DETAILED DESCRIPTION

For better understanding of the present invention, the present invention is illustrated through the following Examples:

### EXAMPLE 1

### Preparation of Hydrogel Tube Having Uniform Size: Acrylic Acid/Acrylamide-Iron Two-Component Chemically-Physically Crosslinked Network Hydrogel Tube

1. Formulation of Hydrogel Prepolymer Solution of Acrylic Acid and Acrylamide. Weighed 4.0 g acrylamide, 0.6 g acrylic acid, 0.008 g N,N-bisacrylamide, and 0.02 g potassium persulfate, dissolved in 40 mL water, and purged with nitrogen for 1 h to remove oxygen.

2. Formation of Hydrogel Film on Iron Wire. An iron wire having a diameter of 1.6 mm was suspensively immersed into a monomer prepolymer solution, removed from the solution after reacted for 10 min (20° C.), aged for 2 h while being isolated from air, and then immersed in ultrapure water to wash by soaking.

3. Physical Coordination Crosslinking in Fe³⁺ Solution. The iron wire having grown gel layer was immersed into a 0.06 mol/L Fe³⁺ solution and soaked for 2 h, then washed by soaking in pure water for 1 h to remove the iron wire template, and thus a hollow hydrogel tube having a tube diameter of 1.8-2.0 mm and a tube wall thickness of 400-1000 μm was obtained, wherein through testing the hollow hydrogel tube had a tensile strength of 1.2-3.0 MPa, which is 1.5 times larger than the original one.

### EXAMPLE 2

### Preparation of Hydrogel Tube Having Uniform Size: Acrylamide/Hydroxyethyl Methacrylate/Polyvinyl Alcohol Composite Network Hydrogel Tube

1. Formulation of Aqueous Solution of Acrylamide/Hydroxyethyl Methacrylate/Polyvinyl Alcohol. Weighed 1 g acrylamide, 6.0 g hydroxyethyl methacrylate, 0.01 g N,N-bisacrylamide, and 0.02 g potassium persulfate, dissolved in 30 mL water, and purged with nitrogen for 1 h to remove oxygen. Then added was a 20 mL aqueous solution of 5% polyvinyl alcohol (polyethylene glycol), stirred uniformly and then placed in an ice-water bath for use.

2. Formation of Hydrogel Film on Iron Wire. An iron wire having a diameter of 1.6 mm was suspensively immersed into an aqueous solution of a mixture of acrylamide/hydroxyethyl methacrylate/polyvinyl alcohol, removed from the solution after reacted for 30 min (20° C.), placed in a freeze dryer (−20° C.), thawed 3 h later (the freezing-thawing process is continued for 3 times), and the iron wire template was removed to obtain a hollow hydrogel tube having a tube diameter of 2.0-3.0 mm and a tube wall thickness of 800-1200 μm.

### EXAMPLE 3

### Preparation of Hydrogel Tube Having Uniform Size: Hydroxyethyl Methacrylate/Sodium Alginate-calcium Composite Network Hydrogel Tube

1. Aqueous Solution of Hydroxyethyl Methacrylate/Sodium Alginate. Weighed 6.0 g, 0.006 g N,N-bisacrylamide, 0.01 g potassium persulfate, dissolved in 50 mL water, added 5.0 g sodium alginate, stirred until the solution became clear, and purged with nitrogen for 1 h to remove oxygen.

2. Formation of Hydrogel Film on Iron Wire. An iron wire having a diameter of 4.0 mm was suspensively immersed into an aqueous solution of hydroxyethyl methacrylate/sodium alginate, removed from the solution after reacted for 30 min (20° C.), aged for 2 h while being isolated from air, and then immersed in ultrapure water to wash by soaking.

3. Physical Coordination Crosslinking in Ca²⁺ Solution. The iron wire having grown gel layer was immersed into a 0.06 mol/L Ca²⁺ solution and soaked for 10 h, then washed by soaking in pure water for 1 h to remove the iron wire template, and thus a hollow hydrogel tube having a tube diameter of 4.5-5.5 mm and a tube wall thickness of 1000-1500 μm was obtained.

### EXAMPLE 4

### Preparation of Hydrogel Tube Having Uniform Size: Hydroxyethyl Methacrylate/Polyethylene Glycol/Bovine Serum Albumin-Tannic Acid Composite Network Hydrogel Tube

1. Aqueous Solution of Hydroxyethyl Methacrylate/Polyethylene Glycol/Bovine Serum Albumin. Weighed 6.5 g hydroxyethyl methacrylate, 0.015 g N,N-bisacrylamide, and 0.01 g potassium persulfate, dissolved in 30 mL water, and purged with nitrogen for 1 h to remove oxygen. Then added was a 20 mL aqueous solution of 5% polyvinyl alcohol and 0.5 g bovine serum albumin, stirred uniformly and then placed in an ice-water bath for use.

2. Formation of Hydrogel Film on Iron Wire. An iron wire having a diameter of 4.0 mm was suspensively immersed into an aqueous solution of a mixture of hydroxyethyl methacrylate/polyethylene glycol/bovine serum albumin, removed from the solution after reacted for 30 min (20° C.), placed in a freeze dryer (−20° C.), thawed 2 h later (the freezing-thawing process is continued for 3 times), and the iron wire template was removed to obtain a hydroxyethyl methacrylate/polyethylene glycol/bovine serum albumin hollow hydrogel tube.

3. Treatment in Tannic Acid Solution. The hydroxyethyl methacrylate/polyethylene glycol/bovine serum albumin hollow hydrogel tube was immersed into an aqueous solution of 3% tannic acid and soaked for 10 h, then washed by soaking in pure water for 5 h, and thus a hydroxyethyl methacrylate/polyethylene glycol/bovine serum albumin-tannic acid composite network hydrogel tube was obtained.

### EXAMPLE 5

### Preparation of Acrylic Acid/Acrylamide-Calcium throughout Hydrogel Tube with Complex Shape

1. Formulation of Hydrogel Prepolymer Solution of Acrylic Acid and Acrylamide. Weighed 3.5 g acrylamide, 0.6 g acrylic acid, 0.003 g N,N-bisacrylamide, and 0.02 g potassium sulfate, dissolved in 40 mL water, and purged with nitrogen for 1 h to remove oxygen.

2. Formation of Hydrogel Film on Iron Wire. Selected iron wires of sizes of 0.3 mm, 0.5 mm, 0.7 mm, 1.2 mm, 1.6 mm, and 4.0 mm were mechanically polished, then coiled and intertwined together, and placed in a specific reaction tank. The monomer prepolymer solution was quickly poured into the reaction tank, removed from the reaction tank after reacted for 10 min (20° C.), aged for 2 h while being isolated from air, and then immersed in ultrapure water to wash by soaking.

3. Physical Coordination Crosslinking in Ga²⁺ solution: the iron wire having grown gel layer was immersed into a 0.2 mol/L Ga²⁺ solution and soaked for 10 h, then washed by soaking in pure water for 2 h to draw out iron wires of different sizes, and thus a through hydrogel tube structure body having a complex shape was obtained.

### EXAMPLE 6

### Preparation of Thickness Gradient Acrylic Acid/Acrylamide-Calcium Hydrogel Tube

1. Formulation of Hydrogel Prepolymer Solution of Acrylic Acid and Acrylamide. Weighed 4.0 g acrylamide, 1.0 g acrylic acid, 0.01 g N,N-bisacrylamide, and 0.02 g potassium persulfate, dissolved in 30 mL water, and purged with nitrogen for 1 h to remove oxygen.

2. Formation of Gradient Hydrogel Film on Iron Wire. An iron wire having a diameter of 1.6 mm was vertically and suspensively fixed onto a drawing machine, quickly immersed into a hydrogel prepolymer solution, slowly pulled out from the solution at a withdrawal rate of 1 cm/min after reacted for 1 min (20° C.), and immersed in ultrapure water to wash by soaking after the full iron wire was totally drawn out from the solution

3. Physical Coordination Crosslinking in Ca²⁺ Solution. The iron wire having grown gel layer was immersed into a 0.06 mol/L Ca²⁺ solution and soaked for 5 h, then washed by soaking in pure water for 1 h to remove the copper wire template, and thus a hollow hydrogel tube having a tube wall thickness distributed gradiently was obtained.

### EXAMPLE 7

### Preparation of Acrylic Acid/Acrylamide-Calcium Hydrogel Tube Having Shape, Crosslinking-Degree and Tube-Wall-Thickness Gradients

1. Formulation of Hydrogel Prepolymer Solution of Acrylic Acid and Acrylamide. Weighed 3.0 g acrylamide, 0.5 g acrylic acid, 0.006 g N,N-bisacrylamide, and 0.01 g potassium persulfate, dissolved in 50 mL water, and purged with nitrogen for 1 h to remove oxygen.

2. Formation of Hydrogel Film on Iron Wire. A metal having a diameter of 1.6 mm was quickly immersed into a hydrogel prepolymer solution, and then immersed in ultrapure water to wash by soaking after reacted for 10 min (20° C.).

3. Physical Coordination Crosslinking in Cu²⁺ Solution. The bottom end of the iron wire having grown gel layer was vertically immersed into a 0.06 mol/L Cu²⁺ solution, wherein the immersed length was about ⅙ of the total length, and the iron wire was drawn out after immersed for 10 h, so as to obtain a hydrogel tube having shape, crosslinking-degree and tube-wall-thickness gradients.

### EXAMPLE 8

### Preparation of Layered (Transverse) Acrylic Acid/Acrylamide-Acrylic Acid/N-Isopropylacrylamide Hydrogel Tube

1. Formulation of Prepolymer Solution A Containing Acrylic Acid/Acrylamide and Prepolymer Solution Containing Acrylic Acid/N-Isopropylacrylamide. Prepolymer Solution A: Weighed 3.0 g acrylamide, 2.0 g acrylic acid, 0.002 g N,N-bisacrylamide, and 0.01 g potassium persulfate, dissolved in 50 mL water, and purged with nitrogen for 1 h to remove oxygen. Prepolymer Solution B: Weighed 6.78 g N-isopropylacrylamide, 1.5 g acrylic acid, 0.002 g N,N-bisacrylamide, and 0.02 g potassium persulfate, dissolved in 30 mL water, and purged with nitrogen for 1 h to remove oxygen.

2. Formation of Hydrogel Film on Iron Wire. An iron wire having a diameter of 1.6 mm was suspensively immersed into a monomer prepolymer solution A, removed from the solution after reacted for 10 min (20° C.), immersed into a monomer prepolymer solution B, removed from the solution after reacted for 30 min (20° C.), and then immersed in ultrapure water to wash by soaking.

3. Post-Enhancement Treatment of Hydrogel Film. The iron wire having layered gel layer grown thereon was immersed into a 0.06 mol/L Ca²⁺ solution and soaked for 5 h, drawn out from the solution to remove the iron wire template, then washed by soaking in pure water for 10 h, and thus a layered hollow hydrogel tube having a tube diameter of 2.0-3.0 mm and a tube wall thickness of 2000-3000 μm was obtained.

4. Description: a variety of hydrogel monomer prepolymer solutions were formulated according to the above steps, and a hydrogel tube having a multi-layer structure was prepared by a continuous immersion growth method.

### EXAMPLE 9

### Preparation of Radial-Gradient Acrylic Acid/Acrylamide Hydrogel Tube

1. Formulation of Hydrogel Prepolymer Solution containing different concentrations of Acrylic Acid/Acrylamide. Prepolymer Solution A: Weighed 3.0 g acrylamide, 2.0 g acrylic acid, 0.003 g N,N-bisacrylamide, and 0.02 g potassium persulfate, dissolved in 10 mL water, and purged with nitrogen for 1 h to remove oxygen. Prepolymer Solution B: Weighed 3.0 g acrylamide, 2.0 g acrylic acid, 0.003 g N,N-bisacrylamide, and 0.02 g potassium persulfate, dissolved in 30 mL water, and purged with nitrogen for 1 h to remove oxygen. Prepolymer Solution C: Weighed 3.0 g acrylamide, 2.0 g acrylic acid, 0.003 g N,N-bisacrylamide, and 0.02 g potassium persulfate, dissolved in 50 mL water, and purged with nitrogen for 1 h to remove oxygen.

2. Formation of Hydrogel Film on Iron Wire. An iron wire having a diameter of 1.6 mm was suspensively immersed into a monomer prepolymer solution A, removed from the solution after reacted for 10 min (20° C.), immersed into a monomer prepolymer solution B, removed from the solution after reacted for 20 min (20° C.), then immersed into a monomer prepolymer solution C, immersed in a 0.06 mol/L Fe³⁺ solution for 1-2 min to remove the iron wire template, and immersed in ultrapure water to wash by soaking, and thus a radial-gradient hydrogel tube was obtained.

What is claimed is:

1. A method for preparing a double-network hydrogel tube with a complex structure, wherein the method comprises the following sequential steps:
1) immersing polished iron wires of different sizes in a monomer prepolymer solution, or pouring a monomer prepolymer solution into a container containing iron wires of different arranged shapes to conduct a polymerization reaction, such that a uniform chemically-crosslinked hydrogel layer is formed on the surface of each iron wire, aging, and then washing by soaking in pure water to obtain a primary-crosslinked single-network hydrogel film; and
2) immersing iron wires having grown hydrogel films in a secondary-crosslinking solution selected from an aqueous solution of calcium ions, an aqueous solution of magnesium ions, an aqueous solution of ferric ions, or an aqueous solution of tannic acid for 5 min to 20 h, and then drawing the iron wires out to obtain high-strength double-network hydrogel tubes of different shapes.

2. The method according to claim 1, wherein the monomer prepolymer solution consists of a monomer, an initiator, an crosslinking agent, an aqueous polymer or biomacromolecule, and high-purity deionized water, wherein the monomer is one or two of (meth)acrylic acid, acrylamide, hydroxyethyl (meth)acrylate, polyoxyethylene methacrylate, N-isopropylacrylamide, methacrylic sulfonate, chitosan (meth)acrylate, chitosan (meth)acrylate, dimethylaminoethyl methacrylate, sodium alginate methacrylate, methylacryloyl ethylcarboxybetaine, and methylacryloyl ethylsulphobetaine; the initiator is potassium persulfate or ammonium persulfate; the crosslinking agent is N,N′-methylene bisacrylamide or (poly)ethylene glycol di(meth)acrylate; the aqueous polymer is polyvinyl alcohol, polyethylene glycol or polyvinylpyrrolidone; and the biomacromolecule is bovine serum albumin, collagen or polypeptide.

3. The method according to claim 2, wherein in the monomer prepolymer solution, the mass fraction of the monomer, the initiator and the crosslinking agent is from 5% to 15%, the molar ratio of the three is 500-1000:1:0.5, the mass fraction of the aqueous polymer is 5-10%, and the balance is deionized water.

4. The method according to claim 2, wherein in the monomer prepolymer solution, the mass fraction of the monomer, the initiator and the crosslinking agent is from 5% to 15%, the molar ratio of the three is 500-1000:1:0.5, the mass fraction of the biomacromolecule is 0.1-1%, and the balance is deionized water.

5. The method according to claim 1, wherein the iron wires are arranged in various ways and may be a single one, arranged in multiple rows, cross-arranged or arranged in an array, so as to obtain a hydrogel tube with a complex shape, and the diameter of the iron wire is 10 μm to 3 mm.

6. The method according to claim 1, wherein the polymerization time is 1 to 30 min and the polymerization temperature is 10° C. to 30° C.

7. The method according to claim wherein the concentration of the secondary-crosslinking solution is 0.1-0.6 mol/L.
